(19) United States
(12) Patent Application Publication
(10) Pub. No.: US 2006/0057170 A1
(43) Pub. Date: Mar. 16, 2006

(54) Oil-in-water emulsion containing fillers

(75) Inventor: Carole Guiramand, Jouy En Josas (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/213,915

(22) Filed: Aug. 30, 2005

Related U.S. Application Data
(60) Provisional application No. 60/615,020, filed on Oct. 4, 2004.

(30) Foreign Application Priority Data
Sep. 13, 2004 (FR) 04/09700

(51) Int. Cl. A61K 8/39

(52) U.S. Cl. 424/401; 424/70.31

(57) ABSTRACT

A composition for topical application in the form of an oil-in-water emulsion containing an oily phase dispersed in an aqueous phase, the globules of oily phase of which have a mean diameter ranging from 50 to 200 nm, prepared without introduction of mechanical or thermal energy and exhibiting good stability and good cosmetic properties. The invention composion can be used in numerous cosmetic and dermatological applications and may be suitable for all skin types and in particular for greasy skin. It can also be used for the treatment of wrinkles.

### REFERENCE TO PRIOR APPLICATIONS

This application claims priority to U.S. provisional application 60/615,020 filed Oct. 4, 2004, and to French patent application 0409700 filed Sep. 13, 2004, both incorporated herein by reference.

### FIELD OF THE INVENTION

The present invention relates to a composition in the form of a oil-in-water (O/W) emulsion comprising fillers. Preferably, the invention composition is one obtained without introduction of energy, and the process of preparation makes up a part of the invention. The invention further relates to the use of the composition, in particular for treating, caring for, making up and/or cleaning the skin, superficial body growths (hair, eyelashes, nails) and/or mucous membranes and more particularly for obtaining a mattifying effect on the skin or for treating wrinkled skin. Preferably, the O/W emulsion of the invention is a fine emulsion. Also preferably, the invention composition is suitable for topical application, and in particular is a cosmetic and/or dermatological composition.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

For various reasons related in particular to better comfort of use (softness, emollience and others), current cosmetic or dermatological compositions are generally provided in the form of an emulsion of the oil-in-water (O/W) type, that is to say a vehicle composed of an aqueous continuous dispersing phase and of an oily discontinuous dispersed phase. These O/W emulsions are in greater demand than emulsions of the water-in-oil (W/O) type, which are composed of a fatty continuous dispersing phase and of an aqueous discontinuous dispersed phase, as they contribute to the skin, on application, a softer, less greasy and lighter feel than W/O emulsion systems.

Furthermore, in the cosmetic, dermatological and pharmaceutical fields, emulsions comprising globules (or droplets) of the dispersed phase which are small in size, also known as fine emulsions, are often desired, whether:

for their texture: the compositions may be more or less viscous and may range from the consistency of a lotion to that of a cream;

for their visual appearance, which may vary from a transparent or opalescent composition to a white composition;

for their cosmetic feel, which promotes in particular their speed of penetration;

for their broad possible positioning in market terms, such compositions being satisfactory to consumers both in Europe and Japan and other countries.

In terms of technology, at the present time, in order to obtain fine O/W emulsions of this type, it is necessary to introduce energy into the mixture, either significant mechanical energy in order to fragment the dispersed phase into fine globules, or thermal energy, involving a process of phase change with temperature, such as the systems prepared by the PIT (Phase Inversion Temperature) technique. These systems are well known and make it possible today to have available fine emulsions.

Thus, the technique of introducing mechanical energy makes it possible to obtain fine transparent emulsions, also known as “nanoemulsions”, for example disclosed in the documents EP-A-728 460, EP-A-780 114, EP-A-780 115, EP-A-879 589, EP-A-1 010 413, EP-A-1 010 414, EP-A-1 010 415, EP-A-1 010 416, EP-A-1 013 338, EP-A-1 016 453, EP-A-1 018 363, EP-A-1 020 219, EP-A-1 025 898, EP-A-1 120 102, EP-A-1 120 101, EP-A-1 160 005, EP-A-1 172 077 and EP-A-1 353 629. The oil globules of nanoemulsions have a mean size of less than 100 nm. The disadvantage of these nanoemulsions is the necessity for the involvement of significant mechanical energy.

Nanoemulsions are also described in the publications by Forgiarini, J. Esquena, C. Gonzalez and C. Solans, “Formation of Nano-emulsions by Low Energy Emulsification. Methods at Constant Temperature”, Langmuir, 2001, 17, 2076-2083H., and by Forgiarini, J. Esquena, C. Gonzalez and C. Solans, “Studies of the Relation between Phase Behavior and Emulsification Methods with Nanoemulsion Formation”, Prog. Colloid Polym. Sci., 2000, 115 (Trends in Colloid and Interface Science XIV), 36-39. These publications describe decane-in-water emulsions stabilized by a specific surfactant, laureth-4 (or Brij 30), and produced by addition of water to a decane/Brij 30 mixture. The surfactant comprises a short (C₁₂) alkyl chain, which renders it more irritating than its homologues comprising a longer alkyl chain. Furthermore, the emulsions described in these documents are unstable, in particular at the microscopic level (diameter of drops), and are for-this reason too unstable for industrial application.

Furthermore, the principle of the PIT technique is well known to a person skilled in the art and the technique is described in particular in the papers “Phase Inversion Emulsification” by Th. Forster et al., which appeared in Cosmetics & Toiletries, vol. 106, December 1991, pp. 49-52 and “Application of the phase-inversion-temperature method to the emulsification of cosmetics” by T. Mitsui et al., which appeared in American Cosmetics and Perfumery, vol. 87, December 1972, and in the documents WO-A-89/11907, DE-A-4 318 171, EP-A-815 846 and EP-A-1 297 824.

However, these techniques for obtaining fine emulsions exhibit the following disadvantages:

the high temperature of the PIT process imposes constraints in terms of formulation. Thus, it is difficult to use this technique with heat-sensitive molecules having low flash points, and the technique is thus limited to heat-insensitive molecules exhibiting high flash points. This restricts the types and the number of starting materials which can be used or else, if it is desired, for example, to use molecules having low flash points, it is necessary, depending on these starting materials, to adjust the procedure and the preparation of these emulsions then becomes more complex and more expensive. For this reason, this process excludes or at least limits the use of volatile compounds, such as volatile lipophilic compounds, in particular volatile oils, such as volatile silicones, and of certain heat-sensitive active principles or plant extracts.

high or very high pressure homogenizers, which make it possible to prepare fine emulsions by introducing energy, are expensive and delicate devices which thus generate high industrial processing costs.

Furthermore, transparent microemulsions are known in the state of the art. Microemulsions are not strictly speaking emulsions, in contrast to nanoemulsions; they are transparent solutions of micelles swollen with oil, this oil being dissolved by virtue of the joint presence of a large amount of surfactants and of cosurfactants forming the micelles. The size of the swollen micelles is very small due to the small amount of oil which they can dissolve. This very small size of the micelles is the cause of their transparency. However, in contrast to the nanoemulsions described above, microemulsions are formed spontaneously by mixing the constituents without introduction of mechanical energy, other than simple magnetic stirring, and whatever the order of addition of the constituents. In addition, these are thermodynamically stable systems. The major disadvantages of microemulsions are related to their high-proportion of surfactants with respect to the oil, leading to intolerance and resulting in a tacky feel when applied to the skin. Furthermore, the microemulsion state of the system is defined by the choice of the constituents and their relative proportions, and also the temperature, as is shown by the phase diagram presented in FIG. 11.7 of the paper “The colloidal domain”, D. F. Evans and H. Wennerstrom, published by Wiley-VCH (1999). Reference may be made, for the description of microemulsions, for example to the paper by M. Bourrel and R. S. Schechter, “Microemulsions and related systems”, pages 25 to 30, published by Marcel Denker, 1988. These microemulsions are therefore not fine emulsions and cannot overcome the disadvantages of the fine emulsions described above.

Furthermore, the fine emulsions have to be able to comprise various additives without being destabilized by this addition. Thus, it is advantageous to add thereto agents which make possible immediate effects on the skin, such as mattifying agents, which make it possible to reduce the shininess of the skin, which is desired in particular for greasy skin, and such as tighteners, which make it possible to obtain a smoothing of the skin and thus an immediate reduction in the wrinkles and fine lines, which is advantageous in particular in the case of mature skin. These agents are generally particles having optical properties.

In point of fact, these compounds often exhibit the disadvantages of being difficult to formulate as they destabilize the emulsion comprising them, this being all the more pronounced when they are introduced into fine emulsions as they exhibit the disadvantage of being systems intrinsically difficult to stabilize.

### SUMMARY OF THE INVENTION

There thus exists a real difficulty in incorporating agents which provide immediate effects on the skin, such as mattifying agents, tighteners, etc., while retaining good stability of the fine emulsions over time and with regard to temperature without the occurrence of destabilization or a change in appearance or a modification in the size of the oil droplets of the fine emulsion.

The need thus remains to produce fine O/W emulsions which remain stable even in the presence of particles and which are obtained with processes which are less expensive and less complex than those of the prior art, that is to say processes not requiring introduction of energy, whether this energy is mechanical-or thermal, and thus without involving high temperatures or without a device introducing a large amount of energy, these processes having no effect on the chemical stability of the compounds constituting the composition.

The inventor has found, surprisingly, that fine emulsions comprising particles can be prepared without introduction of energy and entirely at ambient temperature by virtue of a specific choice of surfactants, of a specific choice of oils, of a specific choice of particles and of a specific oils/surfactant ratio.

### DETAILED DESCRIPTION OF THE PREFERRED EMBODIMENTS

A subject-matter of the present invention is thus a composition, preferably suitable for topical application, in the form of an oil-in-water emulsion comprising an oily phase dispersed in an aqueous phase, the globules of oily phase of which have a mean diameter ranging from 50 to 200 nm, wherein:

it comprises an emulsifying system comprising (i) at least one nonionic surfactant having a melting point of less than 45° C. and a HLB ranging from 10 to 15, the said surfactant comprising a polar part comprising at least 5 oxyethylene groups and a nonpolar part comprising at least one branched or unsaturated alkyl chain having from 14 to 22 carbon atoms, and (ii) at least one anionic surfactant,

the oily phase comprises at least one oily constituent selected from hydrocarbon oils with a molecular weight of equal to or greater than 400, the amount of hydrocarbon oil(s) with a molecular weight of equal to or greater than 400 representing at least 25% by weight with respect to the total weight of the oily phase and the amount of oil(s) based on triglycerides representing less than 15% by weight with respect to the total weight of the oily phase,

the ratio by weight of the amount of the oily constituents of the oily phase to the amount of the emulsifying system ranges from 0.8 to 3.5,

it comprises at least one dispersion of inorganic colloidal particles.

This composition exhibits the inherent advantage of being capable of being prepared without introduction of energy and in particular without introduction of thermal or mechanical energy.

In this context, the term “topical application” is understood to mean the external application to keratinous substances, which are in particular the skin, scalp, eyelashes, eyebrows, nails, hair and/or mucous membranes. As the composition is preferably intended for topical application, it preferably comprises a physiologically acceptable medium. The term “physiologically acceptable medium” is understood to mean a medium compatible with the skin, lips, scalp, eyelashes, eyes, nails and/or hair. The composition can constitute in particular a cosmetic or dermatological composition.

In the present patent application, the term “amount of the oily constituents of the oily phase” is understood to mean the total amount of the oils and other fatty substances present in the oily phase, that is to say the amount of the constituents of oily phase other than the emulsifying system comprising in particular the surfactants and cosurfactants.

In the present patent application, the term “fine emulsion” is understood to mean an emulsion for which the size of the globules of the dispersed phase ranges from 50 to 200 nanometres.

According to a preferred characteristic of the compositions in accordance with the present invention, the mean diameter of the globules (or droplets) of oily phase dispersed in the dispersing aqueous phase ranges from 50 to 200 nm, more particularly from 80 to 200 nm, this mean diameter being a mean intensity diameter measured by quasielastic light scattering, for example with the Model BI-90 device from Brookhaven Instruments Corporation.

Depending on the size of the globules of oily phase, the visual appearance of the composition according to the invention ranges from transparent to a white appearance, passing through opalescent.

The emulsions according to the invention exhibit the advantage of being able to be prepared at ambient temperature and thus by a process which does not decompose the heat-sensitive constituents of the composition (in particular active principles) and which, in addition, is relatively inexpensive, relatively simple and without constraint since no energy is necessary to obtain this system. The emulsions according to the invention additionally exhibit the advantage of being perfectly stable over time and with regard to temperature since no creaming (that is to say, rising of the oil globules), no sedimentation (that is to say, gathering of the oil globules at the bottom of the container) and no phase separation (that is to say, separation of the aqueous and oily phases) are observed over time and at various storage temperatures (4° C., 25° C. and 45° C.). The emulsions according to the invention also exhibit the advantage of being able to be provided in a highly varied manner, that is to say while having a broad range of textures in terms of viscosity or of feel (according to the level of oil, for example), which makes it possible to provide them to consumers having very different habits and sensitivities.

The compositions according to the invention can be more or less viscous and can have an appearance ranging from a lotion (fluid product) to a cream (thick product). Thus, their viscosity can range, for example, from 1 cpoise (1 mPa.s or 0.001 Pa·s) to 200 poises (20 Pa·s), this viscosity being measured after shearing for 10 minutes, using a Rheomat 180 viscometer at a shear rate of 200 rpm, with a spindle suited to the viscosity of the composition.

Colloidal Particles

The term “dispersion of colloidal particles” is understood to mean, within the meaning of the present invention, a dispersion of particles having a number-average diameter of between 0.1 and 100 nm, preferably between 3 and 30 nm, better still between 5 and 20 nm. The dispersing medium is an aqueous, aqueous/alcoholic or alcoholic medium. Preferably, the medium for dispersion of the inorganic colloidal particles is an aqueous, aqueous/alcoholic or alcoholic medium, more preferably an aqueous medium.

These particles retain the abovementioned diameters in the composition comprising them, without aggregating, and thus do not have thickening properties, which means that the composition composed of 15% by weight of colloidal particles according to the invention in water exhibits a viscosity of less than 0.05 Pa·s for a shear rate equal to 10⁻¹ s, the viscosity being measured at 25° C. using a RheoStress RS150 rheometer from Haake in cone/plate configuration, the cone having a diameter of 60 mm and an angle of 2°.

Furthermore, the dispersions of particles according to the invention are preferably transparent, which means that the turbidity of a dispersion of particles at 1% by weight in water is less than 200 NTU, the measurement being carried out at 25° C. using a Hach 2100P turbidometer. The property of the particles of forming a colloidal solution is related to their zeta potential, which is preferably less than −20 mV and more preferably less than −25 mV, at pH 7 and at 25° C., as measured using a Delsa 440SX device from Coulter Scientific Instrument.

The particles used are preferably inorganic particles. Mention may be made, as examples of colloidal particles which can be used according to the invention, for example, of silica, cerium oxide, zirconium oxide, alumina, calcium carbonate, barium sulphate, calcium sulphate, zinc oxide and titanium dioxide, the composite particles comprising them, and their mixtures. Mention may also be made of laponites, such as, in particular, the products sold by Laporte under the name Laponite XLS, Laponite XLG, Laponite RD and Laponite RDS (these products are sodium magnesium silicates and lithium magnesium sodium silicates). Use may also be made of mixtures of various kinds of colloidal particles.

Mention may be made, as aqueous dispersions of titanium dioxide particles, of dispersions of nanosized titanium oxide in water, in particular those sold under the Tioveil names by Uniqema, in particular those sold under the Tioveil AQ names.

Preferably, the inorganic colloidal particles which are preferred according to the invention are silica particles. Colloidal silica particles are available in particular in the form of aqueous dispersions from Catalysts & Chemicals under the trade names Cosmo S-40 and Cosmo S-50.

Mention may also be made of colloidal silica/alumina composite particles. The term “silica/alumina composite” is understood to mean silica particles in which aluminium atoms have been partially substituted for silicon atoms, that is to say particles composed of silicon oxide, the surface of which has been modified chemically so as to replace some at least of the silicon atoms by aluminium atoms, forming at most a monomolecular layer of aluminium. The surface portion of these particles which is covered with aluminium is generally between 1 and 100%, preferably between 1 and 10%, better still between 4 and 6%. These particles can be prepared in particular, as disclosed in the document U.S. Pat. No. 2,892,797, by mixing a silica sol with a sodium aluminate. The aqueous dispersions of these particles can, for example, be those sold by Grace under the references Ludox AM®, Ludox AMX6021®, Ludox AM-SP5B0-10418® and Ludox AM-SP5B0-10419°.

The amount of colloidal particles (as active material) in the composition of the invention generally preferably ranges from 0.01 to 15% by weight and preferably from 0.05 to 10% by weight, with respect to the total weight of the composition.

Emulsifying System

The composition according to the invention comprises an emulsifying system comprising (i) at least one nonionic surfactant and (ii) at least one anionic surfactant. In this emulsifying system, the nonionic surfactant is generally introduced into the oily phase, whereas the anionic surfactant can be introduced either into the aqueous phase or into the oily phase.

The emulsifying system can be present in an amount ranging from, for example, 0.6 to 11% by weight, preferably from 1.1 to 9% by weight, with respect to the total weight of the composition.

1. Nonionic Surfactant

The nonionic surfactants used in the composition of the invention preferably have a melting point of less than 45° C. and are therefore liquid at a temperature of less than 45° C. and in particular they are liquid at a temperature ranging from 20 to 44° C. They have an HLB ranging from 10 to 15 and they comprise a polar part comprising at least 5 oxyethylene groups and a nonpolar part comprising at least one branched or unsaturated alkyl chain having from 14 to 22 carbon atoms.

The HLB (hydrophilic-lipophilic balance) of an emulsifying surfactant is calculated according to the following formula:

$$HLB = 20 \times \frac{hydrophilic w}{total w of the SA}$$

in which hydrophilic w represents the weight of the hydrophilic group (i.e., the polar part) and total w of the SA represents the total weight of the surfactant.

According to a preferred embodiment of the invention, the nonionic surfactants are chosen from polyethylene glycol fatty acid esters, oxyethylenated fatty acid polyol esters, oxyethylenated fatty alcohol ethers and their mixtures. These oxyethylenated surfactants comprise at least 5 oxyethylene groups; they can comprise, for example, from 5 to 21 oxyethylene groups and preferably from 5 to 18 oxyethylene groups.

Mention may be made, as polyethylene glycol fatty acid esters, for example, of PEG-8 isostearate (or polyethylene 400 isostearate), such as the product sold under the name Prisorine 3644 by Uniqema, PEG-10 isostearate, PEG-12 isostearate, PEG-15 isostearate and their mixtures.

Mention may in particular be made, as oxyethylenated fatty acid polyol esters, of oxyethylenated fatty acid glyceryl esters or oxyethylenated fatty acid sorbitol esters, such as, for example, PEG-15 glyceryl isostearate, such as the product sold under the name Oxypon 2145 by Zschimmer Schwarz, PEG-20 sorbitan triisostearate and their mixtures.

Mention may be made, as oxyethylenated fatty alcohol ether, for example, of isosteareth-10, such as the product sold under the name Arosurf 66E10 by Witco.

Use may be made of a mixture of nonionic surfactants.

The amount of nonionic surfactant(s) as are defined above can range, for example, from 0.5 to 10% by weight and preferably from 1 to 8% by weight, with respect to the total weight of the composition.

2. Anionic Surfactant

The anionic surfactants are preferably chosen more particularly from the group formed by:

the alkaline salts of diketyl and dimyristyl phosphate;

the alkaline salts of cholesterol sulphate;

the alkaline salts of cholesterol phosphate;

acylamino acid (or lipoamino acid) salts, such as mono- and disodium acylglutamates, such as the disodium salt of N-stearoyl-L-glutamic acid sold under the name Acylglutamate HS21 by Ajinomoto;

the sodium salts of phosphatidic acids;

alkyl sulphate and alkanesulphonate derivatives;

and their mixtures.

According to a preferred embodiment of the invention, use is made, as anionic surfactant, of an acylamino acid salt, such as mono- and disodium acylglutamates, for example the disodium salt of N-stearoyl-L-glutamic acid sold under the name Acylglutamate HS21 by Ajinomoto.

The anionic surfactants can be present in the emulsion of the invention in an amount preferably ranging from 0.05 to 2% by weight and more particularly from 0.1 to 1% by weight, with respect to the total weight of the composition. Mixtures may be used.

3. Cosurfactants

The emulsifying system can also comprise one or more cosurfactants which are surfactants with an HLB of less than 5. These cosurfactants have a melting point of less than or equal to 45° C. and are thus liquid at a temperature of less than or equal to 45° C.; they are in particular liquid at a temperature of 20 to 45° C. Mention may be made, as cosurfactants, for example, of fatty alcohols, such as isostearyl alcohol or oleyl alcohol, glycerol fatty acid esters, such as glyceryl isostearate, or sorbitan fatty acid esters, such as sorbitan isostearate. The amount of cosurfactants can range, for example, from 0.005 to 5% by weight and preferably from 0.01 to 2% by weight, with respect to the total weight of the composition. Mixtures may be used.

Oily Phase

The oily phase comprises oily constituents and it must comprise at least one hydrocarbon oil with a molecular weight of equal to or greater than 400, the amount of hydrocarbon oils with a molecular weight of equal to or greater than 400 representing at least 25% by weight with respect to the total weight of the oily phase and, for example, from 25 to 100% by weight of the oily phase, better still from 25 to 80% by weight and even better still from 30 to 70% by weight of the oily phase. Furthermore, the oily phase must comprise less than 15% by weight of oils based on triglycerides, with respect to the total weight of the oily phase.

The term “hydrocarbon oil” is understood to mean, in the present patent application, an oil formed essentially, indeed even composed, of carbon and hydrogen atoms and optionally of oxygen or nitrogen atoms and not comprising a silicon or fluorine atom. Such an oil can comprise ester, ether, amine or amide groups. The hydrocarbon oils with a molecular weight of equal to or greater than 400 used according to the invention are chosen from alkanes having a melting point of less than 45° C., fatty acid esters, fatty alcohol ethers and their mixtures. Mention may in particular be made, as hydrocarbon oils with a molecular weight of equal to or greater than 400, of alkanes, jojoba oil, fatty acid esters, such as isoketyl palmitate or isoketyl stearate, oils of vegetable origin or fatty alcohol ethers, such as diisostearyl ether.

The hydrocarbon oils with a molecular weight of equal to or greater than 400 used according to the invention are preferably chosen from alkanes having a melting point of less than 45° C. and in particular from hydrogenated polyisobutene, such as Parleam® oil, liquid petrolatum and their mixtures.

The oils based on triglycerides generally have a molecular weight of greater than 400. However, in order to achieve the aim of the invention, the amount of these oils must be limited to less than 15% by weight and preferably to less than 10% by weight, with respect to the total weight of the oily phase, the oily phase comprising, as indicated above, the oily constituents without the surfactants of the emulsifying system. Mention may be made, as oils based on triglycerides, of oils of vegetable origin, such as sweet almond oil, avocado oil, castor oil, olive oil, sesame oil, groundnut oil, grape seed oil, rapeseed oil, coconut oil, hazelnut oil, shea butter, palm oil, apricot kernel oil, calophyllum oil, rice bran oil, maize oil, wheat germ oil, soybean oil, sunflower oil, evening primrose oil, safflower oil, passionflower oil and rye oil.

In addition to the hydrocarbon oils with a molecular weight of equal to or greater than 400, the oily phase can additionally comprise one or more oils with a molecular weight of less than 400, for example chosen from silicone oils or hydrocarbon oils with a molecular weight of less than or equal to 400. Mention may in particular be made, as silicone oils, of cyclic or linear silicone oils, in particular those having a viscosity of less than or equal to 10 centistokes at 25° C., such as cyclopolydimethylsiloxanes (cyclomethicones), for example cyclopentadimethyl-siloxane and cyclohexadimethylsiloxane. Mention may be made, as hydrocarbon oils having a molecular weight of less than 400, for example, of isoparaffins, such as isododecane (molecular weight: 194), isohexadecane (molecular weight: 258) or dioctylcyclohexane, the fatty acid esters, such as isopropyl myristate, isopropyl palmitate, ethylhexyl palmitate, isostearyl neopentanoate or isononyl isononanoate (molecular weight: 320), or fatty ethers, such as dicaprylyl ether.

The amount of oily phase can range, for example, from 0.5 to 55% by weight with respect to the total weight of the composition, preferably from 1 to 40% by weight, better still from 2 to 40% by weight and even better still from 5 to 30% by weight, with respect to the total weight of the composition.

In the composition according to the invention, the ratio by weight of the amount of the oily constituents of the oily phase to the amount of the emulsifying system ranges from 0.8 to 3.5 and preferably from 0.7 to 3. As indicated above, the amount of the oily constituents of the oily phase corresponds to the total amount of the oils and other fatty substances present in the oily phase, that is to say the amount of the constituents of oily phase other than the surfactants and/or cosurfactants forming part of the emulsifying system.

Aqueous Phase

Conventionally, the aqueous dispersing phase can be composed of water or a mixture of water and of hydrophilic compounds, such as, in particular, polyols (polyhydric alcohols), such as, for example, glycerol, propylene glycol, dipropylene glycol and sorbitol, or water-soluble lower alcohols, such as ethanol, isopropanol or butanol. In addition, it can, of course, comprise water-soluble or water-dispersible adjuvants and in particular the water-soluble cosmetic or dermatological adjuvants conventionally used. Mixtures can be used.

The aqueous phase can represent, e.g., from 45 to 99.5% by weight, with respect to the total weight of the composition, preferably from 60 to 99% by weight, with respect to the total weight of the composition, and better still from 60 to 98% by weight, with respect to the total weight of the composition.

Adjuvants

Mention may in particular be made, among the adjuvants capable of being present in the aqueous phase and/or in the oily phase of the emulsions in accordance with the invention (depending on their water-soluble or fat-soluble nature), of ionic or nonionic thickeners,. antioxidants, emollients, cosmetic or dermatological active principles, fragrances, preservatives, fillers, sequestering agents, pigments, dyes or any other ingredient commonly used in the fields under consideration.

Of course, a person skilled in the art will take care to choose the optional compound or compounds to be added to the composition according to the invention and their amounts so that the advantageous properties intrinsically attached to the composition in accordance with the invention are not, or not substantially, detrimentally affected by the envisaged addition.

Mention may be made, as active principles, for example, of:

moisturizing agents, such as, for example, sodium lactate; polyols and in particular glycerol, sorbitol or polyethylene glycols; mannitol; amino acids; hyaluronic acid; lanolin; urea and mixtures comprising urea, such as NMF (Natural Moisturizing Factor); petrolatum; N-lauroylpyrrolidonecarboxylic acid and its salts; essential fatty acids; essential oils; and their mixtures;

anti-ageing active principles and keratolytic agents, such as a-hydroxy acids and in particular acids derived from fruits, such as glycolic acid, lactic acid, malic acid, citric acid, tartaric acid, mandelic acid, their derivatives and their mixtures; β-hydroxy acids, such as salicylic acid and its derivatives, for example 5-(n-octanoyl)salicylic acid or 5-(n-dodecanoyl)salicylic acid; a-keto acids, such as ascorbic acid or vitamin C and its derivatives, such as its salts, for example sodium ascorbate, magnesium ascorbyl phosphate or sodium ascorbyl phosphate; its esters, such as ascorbyl acetate, ascorbyl palmitate and ascorbyl propionate, or its sugars, such as glycosylated ascorbic acid, and their mixtures; β-keto acids; retinoids, such as retinol (vitamin A) and its esters, retinal, retinoic acid and its derivatives, and also the retinoids disclosed in the documents FR-A-2 570 377, EP-A-199 636, EP-A-325 540 and EP-A-402 072; adapalene; carotenoids; and their mixtures;

vitamins, such as vitamin A and vitamin C indicated above and also such as vitamin E (tocopherol) and its derivatives; vitamin B3 (or vitamin PP or niacinamide) and its derivatives; vitamin B5 (or panthenol or panthenyl alcohol or 2,4-dihydroxy-N-(3-hydroxypropyl)-3,3-dimethylbutanamide), in its various forms: D-panthenol, DL-panthenol, and its derivatives and analogues, such as calcium pantothenate, pantethine, pantetheine or panthenyl ethyl ether, pangamic acid, pyridoxine, pantoyl lactose and the natural compounds comprising the latter, such as royal jelly; vitamin D and its analogues, such as those disclosed in document WO-A-00/26167; vitamin F or its analogues, such as mixtures of unsaturated acids having at least one double bond and in particular mixtures of linoleic acid, of linolenic acid and of arachidonic acid, or compounds comprising them;

antibacterials and anti-seborrhoeic agents, such as salicylic acid, 2,4,4′-trichloro-2′-hydroxy-diphenyl ether (or triclosan), 3,4,4′-trichlorocarb-anilide (or triclocarban), azelaic acid, benzoyl peroxide and zinc salts, such as zinc lactate, gluconate, pidolate, carboxylate, salicylate and/or cysteate.

Mention may in particular be made, as thickeners, of thickening polymers, in particular:

carboxyvinyl polymers, such as the products sold under the names Carbopol (INCI name: carbomer) by Noveon; or polyacrylates and polymethacrylates, such as the products sold under the names Lubrajel and Norgel by Guardian or under the name Hispagel by Hispano Chimica;

polyacrylamides;

optionally crosslinked and/or neutralized polymers and copolymers of 2-acrylamido-2-methyl-propanesulphonic acid, such as the poly(2-acrylamido-2-methylpropanesulphonic acid) sold by Clariant under the name “Hostacerin AMPS” (INCI name: ammonium polyacryldimethyltauramide) or such as crosslinked copolymers of acrylamide and of AMPS which are provided in the form of an W/O emulsion, for example those sold under the name of Sepigel 305 (INCI name: polyacrylamide/C13-14 isoparaffin/laureth-7) and under the name of Simulgel 600 (INCI name: acrylamide/sodium acryloyldimethyltaurate copolymer/isohexadecane/polysorbate 80) by Seppic. Mention may also be made of crosslinked or linear polymers of AMPS comprising a hydrophobic part, such as the products sold under the names Aristoflex SNC, LNC and HMS by Clariant;

polysaccharides, such as xanthan gum, guar gum and its derivatives, such as hydroxypropyl guar, in particular that sold under the name Jaguar HP105 by Rhodia, locust bean gum, gum acacia, scleroglucans, chitin and chitosan derivatives, carrageenans, gellans, alginates or celluloses, such as microcrystalline cellulose, carboxymethylcellulose, hydroxymethyl-cellulose, hydroxypropylcellulose and hydroxyethyl-cellulose, such as the product sold under the name Natrosol 250HHR by Aqualon;

nonionic polymers comprising at least one hydrophobic block and at least one hydrophilic block, such as the polyurethanes sold under the names Serad FX1010, Serad FX1100 (INCI name: steareth-100/PEG-136/HMDI copolymer) and Serad FX1035 by Huls, those sold under the names Rheolate 255, Rheolate 278 and Rheolate 244 by Rheox (INCI name: polyether-urea-polyurethane), those sold under the names DW 1206F, DW 1206J, DW 1206B and DW 1206G by Röhm & Haas (INCI name: polyurethane) and that sold under the name Acrysol RM 2020 by Röhm & Haas. Mention may also be made of the aqueous solutions of copolymer of SMDI and fatty alcohol sold under the names Aculyn 46 (INCI name: PEG-150 stearyl alcohol/SMDI polymer) and Aculyn 44 (INCI name: PEG-150 decyl alcohol/SMDI polymer) by Röhm & Haas;

anionic polymers comprising at least one hydrophobic chain and in particular acrylic or methacrylic polymers or copolymers (including terpolymers) comprising at least one hydrophobic chain, such as copolymers obtained by copolymerization of acrylic or methacrylic acid or their esters with a monomer possessing ethylenic unsaturation comprising a hydrophobic group, such as the crosslinked copolymers sold under the names Pemulen TR1, Pemulen TR2 or Carbopol 1382 (INCI name: acrylates/C10-30 alkyl acrylate crosspolymer) by Noveon; the methacrylic acid/methyl acrylate/ethoxylated alcohol dimethyl(meta-isopropenyl)benzyl isocyanate terpolymer, as a 25% aqueous solution, sold under the name Viscophobe DB1000 by Amerchol, the acrylic acid/oxyethylenated (20 EO) monostearyl itaconate copolymer, as a 30% aqueous dispersion, sold under the name Structure 2001 by National Starch, the acrylic acid/ethoxylated (20 EO) monoketyl itaconate copolymer, as a 30% aqueous dispersion, sold under the name Structure 3001 by National Starch or the acrylic copolymer soluble in an alkaline medium, as a 30% aqueous dispersion, sold under the name Aculyn 22 by Röhm & Haas;

cationic polymers comprising at least one hydrophobic block and at least one hydrophilic block, such as polyquaternium 24, for example the product sold under the name Quatrisoft LM200 by Amerchol;

crosslinked cationic polymers, such as polyquaternium 37 sold under the names Salcare SC96 by Ciba and Synthalen CR by 3V Sigma.

Mention may in particular be made, as fillers, of fibres, for example nylon-6,6 fibres, such as those sold by Paul Bonté.

Another subject-matter of the invention is a process for the preparation of the invention emulsions. This process comprises:

(i) preparing the oily phase (A), comprising the oil or oils or other fatty substances and the emulsifying system (in its entirety or only the nonionic surfactant), with stirring, stirring being carried out, for example, with a magnetic bar, at a temperature ranging approximately from 20° C. to 45° C., until a homogeneous phase is obtained,

(2) introducing, into the phase (A), 0.1 to 3% by weight of water (phase B), with respect to the total weight of the composition, and in mixing until a homogeneous phase (C) is obtained,

(3) adding, to the phase (C), 55 to 75% by weight of water (phase D), with respect to the total weight of the composition, in order to obtain, after mixing, a homogeneous phase (E), and

(4) adding the remainder of the constituents of the aqueous phase (phase F).

Stirring is preferably carried out with a magnetic bar or any other stirring system which gives gentle and thus low-energy stirring, at a temperature which can range from 20° to 45° C. The term “gentle stirring” is understood to mean stirring carried out at a shear rate of less than 1000 s⁻¹ and preferably of less than 100 s⁻¹.

Preferably, fragrance or fragrances can optionally be added at the same time as the phase F, if they are in an amount of less than 0.4% of the total weight of the composition. If they are in greater amounts, the surplus with respect to 0.4% is added to the oily phase. Thus it is that, if the composition comprises 1% of fragrance, 0.6% is in the oily phase and 0.4% is in the aqueous phase (F).

The compositions of the invention can be used on any keratinous substance, such as the skin, scalp, hair, eyelashes, eyebrows, nails or mucous membranes. They can be used as product for caring for the skin, for example as cream for protecting, treating or caring for the face, for the hands or for the body or as body milk for protecting or caring for the skin, scalp or mucous membranes, or as hygiene product, for example as product for cleaning the skin or mucous membranes, or alternatively as hair product or as antisun product.

The compositions can also constitute products for making up the skin and/or hair, for example by incorporating pigments in the composition in order to constitute in particular foundations.

A further subject-matter of the invention is the cosmetic use of the composition as defined above as product for caring for the skin, as hygiene product, as hair product, as antisun product and as make-up product.

Another subject-matter of the invention is a process for the cosmetic treatment of a keratinous substance, such as the skin, scalp, hair, eyelashes, eyebrows, nails, mucous membranes, wherein the composition as defined above is applied to the keratinous substance.

The compositions according to the invention are particularly appropriate as mattifying product for greasy skin or for the treatment of wrinkled skin.

Consequently, another subject-matter of the present invention is a process for the cosmetic treatment of greasy skin, comprising the topical application to the skin of a composition as defined above.

Another subject-matter of the invention is a process for the cosmetic treatment of wrinkled skin, comprising the application to the skin of the composition as defined above.

A further subject-matter of the invention is the cosmetic use of the composition as defined above for smoothing or toning down wrinkles and fine lines and/or retightening the skin.

Furthermore, when the compositions according to the invention have the appropriate fluidity, they can also be used for the impregnation of water-insoluble substrates in order to form articles (such as wipes) intended for caring for, cleaning and/or removing make-up from the skin, eyelashes and/or lips. The water-insoluble substrate can comprise one or more layers and it can be chosen from the group consisting of woven materials, nonwoven materials, foams, sponges or waddings, as sheets, balls or films. It can in particular be a nonwoven substrate based on fibres of natural origin (flax, wool, cotton, silk) or of synthetic origin (cellulose derivatives, viscose, polyvinyl derivatives, polyesters, such as poly(ethylene terephthalate), polyolefins, such as polyethylene or polypropylene, polyamides, such as nylon, or acrylic derivatives). Nonwovens are described generally in Riedel, “Nonwoven Bonding Methods & Materials”, Nonwoven World (1987). These substrates are obtained according to the usual processes of the art of preparing nonwovens.

When the substrate is a nonwoven, use is preferably made of a thick nonwoven which does not roll up into a ball and which is solid enough not to disintegrate and not to pill when applied to the skin. It must be absorbent and soft, at least on one face, for removal of make-up from the eyes in particular. Mention may be made, as appropriate nonwovens, for example, of those sold under the names Ultraloft 15285-01, Ultraloft 182-008, Ultraloft 182-010 and Ultraloft 182-016 by BBA and Vilmed M1519 Blau, Vilmed M 1550 N and 112-132-3 by Freudenberg, that sold under the name Norafin 11601-010B by Jacob Holm Industries or the flocked nonwovens sold under the names Univel 109. and Univel 119 from Uni Flockage.

Furthermore, this substrate can comprise one or more layers having identical or different properties and can have elasticity and softness properties and other properties appropriate for the desired use. The substrates can comprise, for example, two parts having different elasticity properties, as disclosed in the document WO-A-99/13861, or can comprise a single layer with different densities, as disclosed in the document WO-A-99/25318, or can comprise two layers with different textures, as disclosed in the document WO-A-98/18441.

The compositions impregnated on the substrate can comprise any compound appropriate for the desired purpose, for example foaming surfactants, in order to obtain cleaning wipes, or care active principles, in order to obtain wipes for caring for the skin. It is also possible to use them for making up the skin, for example by impregnating the wipe with a composition which comprises pigments and which may constitute a foundation.

Another subject-matter of the invention is thus an article obtained by impregnating a water-insoluble substrate with a composition as defined above.

Another subject-matter of the invention is the use of the composition as defined above in the preparation of an article intended for caring for, removing make-up from, cleaning or making up the skin, lips and/or eyelashes.

The following examples illustrate the invention without exhibiting a limiting nature. The amounts therein are percentages by weight. The compounds are shown under the INCI name or under the chemical name, depending on the circumstances.

### EXAMPLES 1 AND 2 ACCORDING TO THE INVENTION

| | | Example 1 | Example 2 |
|---|---|---|---|
| PEG 8 isostearate | A | 5.80 | 5.80 |
| Hydrogenated | | 8.75 | 8.75 |
| polyisobutene | | | |
| Cyclic silicone | | 8.75 | 8.75 |
| Water | B | 2.18 | 2.18 |
| Water | D | 45.07 | 45.07 |
| Water | F | q.s. for 100 | q.s. for 100 |
| Preservative | | 0.78 | 0.23 |
| Disodium stearoyl- | | 0.16 | 0.16 |
| glutamate | | | |
| Lactic acid | | — | 0.044 |
| Propylene glycol | | 3 | 3 |
| Steareth-100/PEG-136/ | | 0.40 | — |
| HMDI copolymer | | | |
| (Serad FX1100) | | | |
| Cosmos S 40 | | 7 | — |
| Ludox AMX6021 | | — | 7 |
| Fragrance | | 0.1 | 0.1 |
| Diameter of the | 24 h | 91 nm | 91 nm |
| globules of the oily | | | |
| phase* | | | |
| Viscosity at 25° C. | | 2.6 poises | Liquid |
| (Rheomat 180) | | (0.26 Pa · s) | (approximately |
| | | | 100 cpoises = 0.1 mPa · s) |
| Visual appearance | | Opalescent | Opalescent |
| | | fluid | fluid |
| | | composition | composition |
| Stability at various | | Stable** | Stable** after |
| temperatures (4°, AT, | | after 60 days | 21 days |
| 45° C.) | | | |

*the diameter measured by quasielastic light scattering, the measurement being carried out with a Brookhaven BI-90. For Example 1, the diameter of the globules of oily phase is measured before gelling by the polymer.
**stable means that no creaming, no sedimentation and no phase separation occurs and that the formulation remains in accordance with the physicochemical characteristics found at 24 hours.

### EXAMPLE 3 ACCORDING TO THE INVENTION AND COMPARATIVE EXAMPLE

| | | Example 3 | |
|---|---|---|---|
| | | according to | Comparative |
| | | the invention | Example |
| PEG 8 isostearate | A | 5.80 | 5.80 |
| Hydrogenated | | 8.75 | 8.75 |
| polyisobutene | | | |
| Cyclic silicone | | 8.75 | 8.75 |
| Water | B | 2.18 | 2.18 |
| Water | D | 45.07 | 45.07 |
| Water | F | q.s. for 100 | q.s. for 100 |
| Preservative | | 0.78 | 0.78 |
| Disodium stearoyl- | | 0.16 | 0.16 |
| glutamate | | | |
| Propylene glycol | | 3.00 | 3.00 |
| Steareth-100/PEG-136/ | | 0.40 | 0.40 |
| HMDI copolymer | | | |
| Aqueous dispersion of | | 6.25 | — |
| nanosized titanium | | | |
| oxide (Tioveil AQ G) | | | |
| (comprising 40% of | | | |
| active material) | | | |
| Nanosized titanium | | — | 2.5 |
| oxide treated with | | | |
| aluminium stearate | | | |
| (Micro TiO₂ MT 100 Z) | | | |
| (comprising 100% of | | | |
| active material) | | | |
| Macroscopic appearance | 24 h | Homogeneous | Nonhomogeneous |
| | | white fluid | white fluid |
| | | Particles not | Particles |
| | | visible | visible and |
| | | | concentrated |
| | | | at the surface |
| | | | a few hours |
| | | | after |
| | | | manufacture |
| Viscosity at 25° C. | | 22 poises | Not measured |
| (Rheomat 180) | | | (product non- |
| | | | homogeneous) |
| pH (25° C.) | | 7.8 | 7.8 |
| Size obtained after | | 254 nm | Not measured |
| addition of the | | | (product non- |
| nanosized titanium | | | homogeneous) |
| oxide (measured with a | | | |
| Brookhaven BI-90) | | | |
| Stability at various | | After 15 | After 15 days, |
| temperatures (4°, AT, | | days: stable | layer of |
| 45° C.) | | | particles |
| | | | visible in the |
| | | | bottom of the |
| | | | flask |
| | | | (sedimentation |
| | | | of the |
| | | | particles). |

The comparative example shows that the introduction of nanosized titanium oxides not provided in the dispersion form does not make it possible to obtain a homogeneous and stable composition whereas the aqueous dispersions give homogeneous and stable compositions.

The above written description of the invention provides a manner and process of making and using it such that any person skilled in this art is enabled to make and use the same, this enablement being provided in particular for the subject matter of the appended claims, which make up a part of the original description and including a composition for topical application in the form of an oil-in-water emulsion comprising an oily phase dispersed in an aqueous phase, the oil globules of which have a mean diameter ranging from 50 to 200 nm, wherein:

it comprises an emulsifying system comprising (i) at least one nonionic surfactant having a melting point of less than 45° C. and a HLB ranging from 10 to 15, the said surfactant comprising a polar part comprising at least 5 oxyethylene groups and a nonpolar part comprising at least one branched or unsaturated alkyl chain having from 14 to 22 carbon atoms, and (ii) at least one anionic surfactant,

the oily phase comprises oily constituents including at least one hydrocarbon oil with a molecular weight of equal to or greater than 400, the amount of hydrocarbon oils with a molecular weight of equal to or greater than 400 representing at least 25% by weight with respect to the total weight of the oily phase and the amount of oils based on triglycerides representing less than 15% by weight with respect to the total weight of the oily phase,

the ratio by weight of the amount of the oily constituents of the oily phase to the amount of the emulsifying system ranges from 0.8 to 3.5,

it comprises at least one dispersion of inorganic colloidal particles.

As used above, the phrases “selected from the group consisting of,” “chosen from,” “selected from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out. Terms such as “contain(s)” and the like as used herein are open terms meaning ‘including at least’ unless otherwise specifically noted.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition in the form of an oil-in-water emulsion comprising an oily phase dispersed in an aqueous phase, the oil globules of which have a mean diameter ranging from 50 to 200 nm, wherein said composition comprises an emulsifying system comprising
(i) at least one nonionic surfactant having a melting point of less than 45° C. and a HLB ranging from 10 to 15, the said surfactant comprising a polar part comprising at least 5 oxyethylene groups and a nonpolar part comprising at least one branched or unsaturated alkyl chain having from 14 to 22 carbon atoms, and
(ii) at least one anionic surfactant,
and wherein:
the oily phase comprises at least one oily constituent selected from hydrocarbon oils with a molecular weight of equal to or greater than 400, the amount of hydrocarbon oils with a molecular weight of equal to or greater than 400 representing at least 25% by weight with respect to the total weight of the oily phase and the amount of oils based on triglycerides representing less than 15% by weight with respect to the total weight of the oily phase,
the ratio by weight of the amount of the oily constituents of the oily phase to the amount of the emulsifying system ranges from 0.8 to 3.5,
the composition comprises at least one dispersion of inorganic colloidal particles.

2. The composition according to claim 1, wherein the nonionic surfactant is chosen from polyethylene glycol fatty acid esters, oxyethylenated fatty acid polyol esters, oxyethylenated fatty alcohol ethers and their mixtures.

3. The composition according to claim 1, wherein the nonionic surfactant is chosen from PEG-8 isostearate, PEG-10 isostearate, PEG-12 isostearate, PEG-15 isostearate, PEG-15 glyceryl isostearate, PEG-20 sorbitan triisostearate, isosteareth-10 and their mixtures.

4. The composition according to claim 1, wherein the amount of nonionic surfactant(s) ranges from 0.5 to 10% by weight, with respect to the total weight of the composition.

5. The composition according to claim 1, wherein the anionic surfactant is selected from the group consisting of:
the alkaline salts of diketyl and dimyristyl phosphate;
the alkaline salts of cholesterol sulphate;
the alkaline salts of cholesterol phosphate;
acylamino acid salts;
the sodium salts of phosphatidic acid;
alkyl sulphates and alkanesulphonates;
and their mixtures.

6. The composition according to claim 1, wherein the amount of anionic surfactant(s) ranges from 0.05 to 2% by weight, with respect to the total weight of the composition.

7. The composition according to claim 1, wherein the amount of emulsifying system ranges from 0.6 to 11% by weight, with respect to the total weight of the composition.

8. The composition according to claim 1, wherein the particles are in an aqueous, aqueous/alcoholic or alcoholic medium.

9. The composition according to claim 1, wherein the particles have a number-average diameter of between 0.1 and 100 nm.

10. The composition according to claim 1, wherein the particles have a number-average diameter of 3-30 nm.

11. The composition according to claim 1, wherein the particles are chosen from silica, cerium oxide, zirconium oxide, alumina, calcium carbonate, barium sulphate, calcium sulphate, zinc oxide, titanium dioxide, the composite particles comprising them, laponites and their mixtures.

12. The composition according to claim 1, comprising at least one of colloidal silica particles and colloidal silica/alumina composite particles.

13. The composition according to claim 1, wherein the amount of colloidal particles ranges from 0.01 to 15% by weight, with respect to the total weight of the composition.

14. The composition according to claim 1, wherein the hydrocarbon oil with a molecular weight of equal to or greater than 400 is chosen from alkanes having a melting point of less than 45° C., fatty acid esters, fatty alcohol ethers and their mixtures.

15. The composition according to claim 1, comprising an alkane chosen from hydrogenated polyisobutene, liquid petrolatum and their mixtures.

16. The composition according to claim 1, wherein the amount of oily phase ranges from 0.5 to 55% by weight, with respect to the total weight of the composition.

17. The composition according to claim 1, wherein it is prepared without introduction of energy.

18. A process for the cosmetic treatment of a keratinous substance, comprising applying the composition of claim 1 to the keratinous substance.

19. An article obtained by impregnation of a water-insoluble substrate with a composition according to claim 1.

20. A process for the preparation of the composition according to claim 1, comprising:
(1) preparing oily phase (A), comprising the oil or oils and other fatty substances and the emulsifying system, with stirring, at a temperature ranging from 20° C. to 45° C., until a homogeneous phase is obtained,
(2) introducing, into phase (A), 0.1 to 3% by weight of water (phase B), with respect to the total weight of the composition, and in mixing until a homogeneous phase (C) is obtained,
(3) adding, to phase (C), 55 to 75% by weight of water (phase D), with respect to the total weight of the composition, in order to obtain, after mixing, a homogeneous phase (E), and
(4) adding any remainder of the constituents of the aqueous phase (phase F).
